(19) United States
(12) Patent Application Publication
(10) Pub. No.: US 2026/0177465 A1
(43) Pub. Date: Jun. 25, 2026

(54) Method and device for foundation bearing capacity test based on physical modeling

(71) Applicant: CHINA INSTITUTE OF WATER RESOURCES AND HYDROPOWER RESEARCH, BEIJING (CN)

(72) Inventors: Penghai YIN, BEIJING (CN); Shu YU, BEIJING (CN); Zuyu CHEN, BEIJING (CN); Yijie WANG, BEIJING (CN); Jianbin XU, BEIJING (CN); Naixin WANG, BEIJING (CN); Xuedong ZHANG, BEIJING (CN); Jing HU, BEIJING (CN); Jintong WANG, BEIJING (CN); Yifan ZHANG, BEIJING (CN); Yusheng YANG, BEIJING (CN); Ziqi YAN, BEIJING (CN)

(73) Assignee: CHINA INSTITUTE OF WATER RESOURCES AND HYDROPOWER RESEARCH, BEIJING (CN)

(21) Appl. No.: 19/396,206

(22) Filed: Nov. 20, 2025

(30) Foreign Application Priority Data
Dec. 19, 2024 (CN) 202411884865.9

(51) Int. Cl. G01N 3/16; G01N 1/28; G01N 1/30; G01N 1/38

(52) U.S. Cl. CPC G01N 3/165; G01N 1/286; G01N 2203/0218; G01N 2203/027; G01N 2203/0641

(57) ABSTRACT

The present invention discloses a method and device for a foundation bearing capacity test based on physical modeling, which belongs to the field of foundation bearing capacity tests. The method comprises: S1, taking foundation soil materials and a measurer; S2, preparing a foundation structure in the model box and fixing the measurer in a predetermined position; S3, providing a strip base on the surface or inside of the foundation structure; S4, providing a load applying device in the model box and rigidly connecting the same to the strip base so as to apply a load; S5, hoisting and fixing the model box in the centrifuge, applying a centrifugal force according to a predetermined flow, and collecting the measurement results of each measurer; after reaching a preset condition, applying a load on the foundation structure until a target termination condition is reached, and collecting relevant data; and S6, ending the test. The present invention can achieve the similarity of the mechanical characteristics of the reduced-scale physical model by means of physical modeling test, providing a unique and effective foundation bearing capacity model test method, and thus effectively improving the reliability of the results of the foundation bearing capacity model test.

### CROSS-REFERENCE TO RELATED APPLICATION

This application claims the priority benefit to China application CN202411884865.9 filed on Dec. 19, 2024, the content of which is herein incorporated by reference in their entirety and made a part of this specification.

### TECHNICAL FIELD

The present invention belongs to the technical field of foundation bearing capacity tests, and particularly relates to a method and device for a foundation bearing capacity test based on physical modeling.

### BACKGROUND ART

With the continuous improvement of engineering construction on foundation evaluation and treatment technical requirements, the accurate determination of foundation bearing capacity of different geotechnical materials has become a key technical problem to be solved urgently. The existing mathematical models of foundation bearing capacity calculation is required to be verified by physical model test results. Thus, the physical model test plays an irreplaceable role in the field of foundation bearing capacity research. The commonly used scale physical model test is limited by the size effect, and often cannot simulate the real stress state of the actual foundation, which cannot accurately reflect the bearing capacity level of the actual foundation, resulting in errors in the test results. In the prior art, the artificial centrifugal force is also used to simulate the actual foundation gravity, but it fails to provide an effective test method for determining the foundation bearing capacity under the conditions of complex geology and groundwater level, resulting in that the accurate and reliable results cannot be obtained in the existing foundation bearing capacity test.

### SUMMARY OF THE INVENTION

The present invention has been made to solve at least one of the above-mentioned technical problems occurring in the related art to some extent.

Therefore, it is an object of the present invention to provide a method and device for a foundation bearing capacity test based on physical modeling, which can realize the foundation bearing capacity test by means of physical modeling, provide a unique and effective test process, and effectively improve the accuracy of the result of foundation bearing capacity test.

In order to solve the above-mentioned technical problem, the present invention is realized as follows.

An embodiment of the present invention provides a method for a foundation bearing capacity test based on physical modeling, wherein the method comprises the steps of:
S1, taking a certain amount of foundation soil materials, and a measurer for the test;
S2, preparing a foundation structure to be measured in a model box by the foundation soil materials; fixing each measurer in a predetermined position in or near the foundation structure during the preparation of the foundation structure;
S3, providing a strip base on the surface of the foundation structure or at a certain buried depth;
S4, providing a bearing capacity pressing device in the model box, and rigidly connecting a pressing head of the bearing capacity pressing device to the strip base so as to apply a load to the foundation structure via the strip base during the test;
S5, hoisting and fixing the model box in a basket of a centrifuge, operating the centrifuge according to a pre-set flow to apply a centrifugal force to the model box, and recording the measurement results of each relevant measurer via a data automatic collection system; applying a load to the foundation structure by the bearing capacity pressing device until a target termination condition after reaching a predetermined condition, and automatically recording load-related data and the like; and
S6, stopping the centrifuge to end the test, and analyzing relevant data recorded during the test to determine the bearing capacity of the foundation structure.

In addition, the method for the foundation bearing capacity test based on physical modeling according to the present invention may further have the following additional technical features.

In some of these implementations, when the foundation structure is prepared in the step S2, the foundation structure is prepared by filling the foundation structure in a plurality of layers in order from bottom to top; after each layer is filled and smoothed, a groove with an about 5 mm depth is created by removing a corner of the layer on a side adjacent to the observation window; and then uniform fine sand dyed white is filled into the groove to form a color line mark for observation of deformation of the foundation structure.

In some of these implementations, after the foundation structure is prepared, water is uniformly supplied from the bottom of the foundation structure by a water supply system until the water level in the foundation structure reaches a predetermined water level to simulate different ground water level conditions.

In some of these implementations, the measurer comprises a plurality of laser ranging displacement sensors, a set of particle image velocimetry equipment, a plurality of earth pressure gauges and a plurality of pore water pressure gauges;
the laser ranging displacement sensor is provided above the foundation structure and is configured for measuring displacement data on the surface of the foundation structure;
the particle image velocimetry equipment is positioned directly opposite the foundation structure, and image data of the foundation structure is collected by an observation window of the model box and a color line mark is identified, so as to realize the measurement of soil body displacement velocities at different positions of the foundation structure;
the earth pressure gauge is provided at the very bottom of the foundation structure for measuring the pressure which the foundation structure has under the corresponding environment; and
the pore water pressure gauge is provided inside the foundation structure, and is configured for monitoring the change of pore water pressure in the process of increasing centrifugal acceleration and in the process of pressing and loading by the bearing capacity pressing device.

In some of these implementations, in the step S5, according to the contents of operating the centrifuge to apply a centrifugal force to the model box according to a predetermined flow, at the beginning of the test, the centrifugal acceleration is increased step by step according to the increment of n times of gravity acceleration, and the centrifugal acceleration is loaded several times to the preset target value, n being a positive integer; after the acceleration of each stage is loaded to a set value, the earth pressure gauge in the measurer is read and analyzed; after the reading of the earth pressure gauge is stable, it continues to operate for a predetermined period of time, reading the reading of the laser ranging displacement sensor in the gauge, and determining the surface displacement of the foundation structure in the vertical direction; and the above steps are cycled until the pre-set centrifugal acceleration target value is loaded.

In some of these implementations, in the step S5, according to the content of applying a load to the foundation structure by the bearing capacity pressing device,
the bearing capacity pressing device performs a pressing operation in an intermittent stepped loading manner, each pressing applied on the strip base provided on the foundation structure at a first pressing speed; after pressing to a first pressure threshold value, the pressing head stops pressing until the vertical settlement displacement of the strip base reaches a relatively stable value and the measured value of the pore water pressure gauge returns to the numerical value before loading; and then the next step of loading is continued in the above-mentioned manner until the pressing operation ends when the vertical displacement of the strip base reaches the first displacement threshold value.

An embodiment of the present invention also provides a device for a foundation bearing capacity test based on physical modeling, wherein the test device comprises:
a model box provided in a basket of the centrifuge and configured for providing a model space and a measurement space of a plurality of measurers for the foundation bearing capacity test;
a foundation structure provided inside the model box and configured for a model to be tested for foundation bearing capacity test;
a strip base provided on an upper surface or at a certain buried depth of the foundation structure, being in contact with and connected to the foundation structure, and configured for bearing the load applied by the pressing device and transmitting same to the foundation structure, so as to realize the foundation bearing capacity test;
a bearing capacity pressing device which is rigidly connected to the strip base and configured for providing a load for the foundation bearing capacity test;
a centrifuge configured for fixing the model box and applying a centrifugal force to the foundation structure therein, so as to simulate a measurement environment of a prototype foundation equivalent to several times of the self-weight of the foundation structure; and
a plurality of measurers respectively provided inside or around the foundation structure for measuring various parameters of the foundation structure before the test, during the test and after the test is finished.

In some of these implementations, the bearing capacity pressing device is a hydraulic device.

In addition, the device for the foundation bearing capacity test based on physical modeling according to the present invention may further have the following additional technical features.

In some of these implementations, the foundation structure is prepared in layers, and a dyed sand marker band is provided between each layer; and
according to different water level control conditions in the foundation structure, the foundation structure is a sand foundation or a viscous foundation under a saturated working condition, or the foundation structure is a sand foundation or a viscous foundation under an unsaturated working condition.

In some of these embodiments, the relationship between the pre-set target centrifugal acceleration of the centrifuge and each parameter of the foundation structure comprises:

$$\begin{matrix}\frac{{L}_{p}}{{L}_{m}} = N \\ \frac{{H}_{p}}{{H}_{m}} = N \\ \frac{{σ}_{p}}{{σ}_{m}} = 1\end{matrix}$$

wherein, in the formula,
N represents that the pre-set target value of centrifugal acceleration is N times of the gravity acceleration;
Lₚ and Lₘ are a base width of the prototype foundation and a base width of the foundation structure, respectively;
Hₚ and Hₘ are a foundation depth of the prototype foundation and a foundation depth of the foundation structure, respectively; and
σₚ and σₘ are a foundation soil pressure of the prototype foundation and a foundation soil pressure of the foundation structure, respectively.

In some of these implementations, the measurer comprises a plurality of laser ranging displacement sensors, a set of particle image velocimetry equipment, a plurality of earth pressure gauges and a plurality of pore water pressure gauges;
the laser ranging displacement sensor is provided above the foundation structure and is configured for measuring displacement data on the surface of the foundation structure;
the particle image velocimetry equipment is positioned directly opposite the foundation structure, and image data of the foundation structure is collected by an observation window of the model box and a color line mark is identified, so as to realize the measurement of soil body displacement velocities at different positions of the foundation structure;
the earth pressure gauge is provided at the very bottom of the foundation structure for measuring the pressure which the foundation structure has under the corresponding environment; and
the pore water pressure gauge is provided inside the foundation structure, and is configured for monitoring the change of pore water pressure in the process of increasing centrifugal acceleration and in the process of pressing and loading by the bearing capacity pressing device.

Compared to the prior art, the present invention has at least the following beneficial effects.

In an embodiment of the present invention, the method for the foundation bearing capacity test based on physical modeling is provided to provide a complete and reliable foundation bearing capacity test method, which can accurately measure the bearing capacity of a sand foundation or a cohesive soil foundation under saturated conditions and unsaturated conditions, and provide reliable experimental data for determining the bearing capacity of a prototype foundation under different strata and groundwater levels.

In an embodiment of the present invention, according to the method for the foundation bearing capacity test based on physical modeling, particle image velocimetry equipment and color line marking are used to realize the displacement information of soil at different depths in the foundation structure during the foundation bearing capacity test. At the same time, a laser ranging displacement sensor provided above the foundation structure is used to realize more accurate measurement of the surface displacement data of the foundation structure. The combination of the two measurement methods can bring more reliable displacement data of the foundation structure, and improve the accuracy of the foundation bearing capacity test.

In an embodiment of the present invention, the method for the foundation bearing capacity test based on physical modeling is provided to simulate the natural deposition and self-weight consolidation processes of foundation geotechnical materials by applying centrifugal acceleration to a pre-set target value step by step. A load is applied by the pressing heads in stages, each stage of the load is maintained for a period of time after being applied until the vertical settlement displacement of the strip base reaches a relative stability, and then the next stage of the load is applied. The change of the measured value of the pore water pressure during the loading process is confirmed so as to eliminate the effect of the excess pore water pressure on the test result of the foundation bearing capacity of the foundation structure. The above-mentioned loading methods provide a reliable and effective test method for the foundation bearing capacity test of the present invention, and can bring more accurate and effective measurement data than other loading methods.

The device for a foundation bearing capacity test based on physical modeling of the present invention is used to implement the method for the foundation bearing capacity test based on the physical modeling, and thus has at least all the features and advantages of the method for the foundation bearing capacity test based on the physical modeling, which will not be described in detail herein. Additional aspects and advantages of the invention will be set forth in part in the description which follows and, in part, will be obvious from the description, or may be learned by practice of the invention.

### BRIEF DESCRIPTION OF THE DRAWINGS

FIG. 1 is a flow chart of a method for a foundation bearing capacity test based on physical modeling according to an embodiment of the present invention;

FIG. 2 is a real picture of a 450 g-ton large geotechnical centrifuge according to an embodiment of the present invention;

FIG. 3 is a structurally schematic view of a model box of a centrifuge according to an embodiment of the present invention;

FIG. 4 is a structure view of a foundation structure model according to an embodiment of the present invention; wherein (a) is a front view, and (b) is a top view;

FIG. 5 is a structure view of a strip base according to an embodiment of the present invention; wherein (a) is a front view, and (b) is a top view;

FIG. 6 is a schematic view of hydraulic loading according to an embodiment of the present invention;

FIG. 7 is an exploded view of a connection portion of a hydraulic loading device according to an embodiment of the present invention;

FIG. 8 is a structurally schematic plan view of a hydraulic loading device according to an embodiment of the present invention;

FIG. 9 is a state diagram after the completion of the hydraulic loading according to an embodiment of the present invention;

FIG. 10 is a front view of a measurer arrangement according to an embodiment of the present invention; and

FIG. 11 is a top view of a measurer arrangement according to an embodiment of the present invention.

### DETAILED DESCRIPTION OF THE INVENTION

The technical solutions in the embodiments of the present invention will be described clearly and completely in conjunction with the accompanying drawings in the embodiments of the present invention. Obviously, the described examples are part of the examples of the present invention, rather than all of the embodiments. Based on the embodiments of the present invention, all other embodiments obtained by those of ordinary skill in the art without creative efforts shall fall within the protection scope of the present invention.

The following provides a detailed description of the embodiments of the present invention in combination with the accompanying drawing by specific embodiments and their application scenarios.

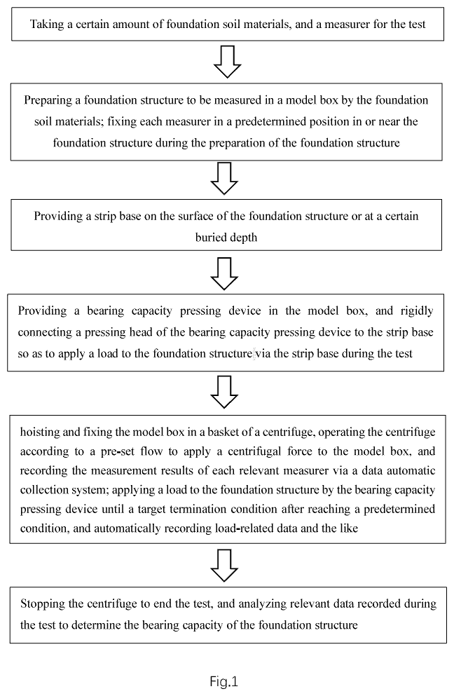

Referring to FIG. 1, in some embodiments of the present invention, there is provided a method for a foundation bearing capacity test based on physical modeling, in which the centrifugal acceleration is applied to a foundation structure made of a limited amount of geotechnical materials by a centrifuge to load a measurement environment of the foundation structure so as to simulate a real stress state of the foundation, thereby accurately determining the foundation bearing capacity. The test procedure for determining the foundation bearing capacity using the physical modeling test method is an innovative point of the present invention. The specific content of the foundation bearing capacity test method based on the physical modeling of the present invention will be described below.

### 1 Description of Test Design

### 1.1 Test Principles

In the centrifugal model test method, the centrifugal field is used to improve the model's bulk force and form artificial gravity. When the ratio of prototype size to model size is n, the pre-set target value aₘ of centrifugal acceleration is

$${a}_{m} = \frac{{L}_{p}}{{L}_{m}} g = ng$$

In the formula, Lₚ is the prototype size; Lₘ is the size of model; and g is the acceleration of gravity.

When the prototype size 1/n test model is placed in the ng centrifugal gravity field and the self-weight of the test model is increased by n times, the stress of each point in the model is the same as that of the corresponding point in the prototype, which shows the similarity law of centrifugal model test. Table 1 lists the similarity law for the relevant parameters of the centrifugal model test.

TABLE 1
Similarity law for relevant parameters of centrifugal model test

| Characteristic | Dimension | Ratio of model to prototype |
|---|---|---|
| Length | L | 1:n |
| Area | L² | 1:n² |
| Volume | L³ | 1:n³ |
| Force | F | 1:n² |
| Stress | FL⁻² | 1:1 |
| Strain | — | 1:1 |
| Displacement | L | 1:n |
| Density | ML⁻³ | 1:1 |
| Compressibility factor | L²F⁻¹ | 1:1 |
| Internal friction angle | — | 1:1 |
| Cohesive force | ML⁻¹T⁻² | 1:1 |
| Inertial acceleration | LT⁻² | n:1 |
| Self-weight stress | ML⁻¹T⁻² | 1:1 |
| Bearing capacity | ML⁻¹T⁻² | 1:1 |
| Compression modulus | ML⁻¹T⁻² | 1:1 |
| Pore pressure | ML⁻¹T⁻² | 1:1 |
| Settling | L | 1:n |
| Time | T | 1:n² |

### 1.2 Purpose of Test

The purpose of this test is to comprehensively evaluate the influence of groundwater level fluctuation on the bearing capacity and settlement behavior of foundation structure. The change of groundwater level and load on the foundation structure are simulated by the centrifugal model test to explore the mechanical response of the foundation structure under different water levels, so as to provide important theoretical and data support for the design and maintenance of urban infrastructure such as subway.

### 1.3 Working Conditions

The LXJ-4-450 large-scale geotechnical centrifuge is used in the test. The maximum rotating radius of the centrifuge is 5.03 m. The maximum acceleration is 300 g. The effective load is 1.5 tons. The effective load capacity is 450 g-ton. The DC motor is used to drive the centrifuge. The power of the DC motor is 700 kW. The size of centrifuge basket is 1.5 m×1.0 m×1.5 m. The performance indicators of this centrifuge are shown in Table 2.

TABLE 2
Performance indicators of LXJ-4-450
large-scale geotechnical centrifuge

| Items | Parameters | |
|---|---|---|
| Effective load capacity (g-capacity) | 450 | |
| Maximum rotating radius (m) | 5.03 | |
| Maximum acceleration (g) | 300 | |
| Effective load (t) | 1.5 | |
| Test basket size(m × m × m) | 1.5 × 1.0 × 1.5 | |
| Structural form | Symmetric arm, double basket | |
| Continuous running time | 48 | h |
| Motor power | 700 | kW |

### 1.4 Test Device Preparation

### 1.4.1 Centrifugal Test Model Size

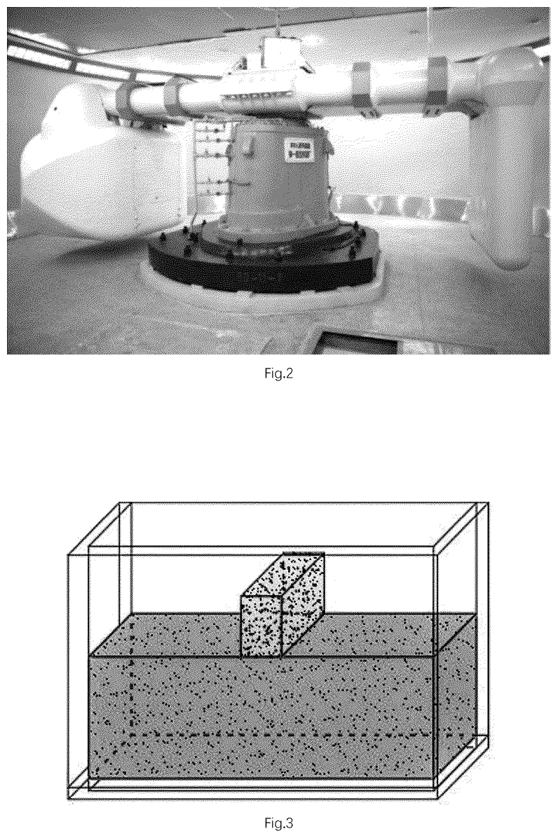

The pre-set target centrifugal acceleration of the centrifugal model is 50 g. The size of the experimental model box is 850 mm (inner length)×305 mm (inner width)×573 mm (inner height). The total mass of the model box is 108 kg. In the experiment, a hydraulic loading device and a strip base are designed, as shown in FIGS. 2 and 3.

### 1.4.2 Total Mass of Soil Body

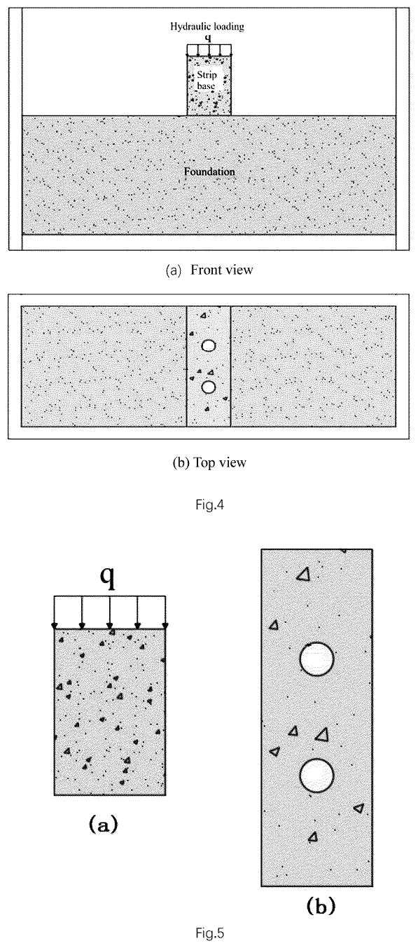

As shown in FIG. 4, the area of the foundation structure model can be calculated to be 2,592.5 cm². The width of the model is 305 mm, and the volume of the model can be calculated to be 77,775 cm³. The foundation material of this test is Fujian standard sand. The dry density of the sand soil under the condition of 75% relative density is 1,584 kg/m³, and the total mass of the unearthed soil is 125.25 kg.

### 1.4.3 Total Mass of Strip Base

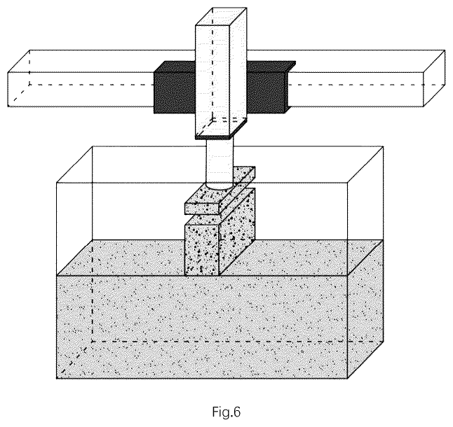

As shown in FIG. 5, the size of the strip base is 305 mm (length)×100 mm (width)×150 mm (height), and the buried depth in the soil body is 50 mm. The volume of the strip base is 4,575 cm³. The density of concrete is 2,400 kg/m³, and the mass of strip base is calculated as 10.98 kg.

### 2 Centrifugal Model Test Method for Foundation Bearing Capacity

### 2.1 Total Mass of Test Model

$$Total mass of test model = mass of model box ( 108 kg ) + mass of soil body ( 125.25 kg ) + mass of strip base ( 10.98 kg ) + weight of reaction frame and cross beam ( 500.33 kg ) = 744.56 kg .$$

### 2.2 Similarity Criterion

Assuming that the pre-set target value of centrifugal acceleration is N times of gravity acceleration, i.e., N=prototype size/model size, the scale relationship of model is as follows:

$$\frac{{L}_{p}}{{L}_{m}} = N \qquad ( 2 - 1 )$$

$$\frac{{H}_{p}}{{H}_{m}} = N \qquad ( 2 - 2 )$$

$$\frac{{σ}_{p}}{{σ}_{m}} = 1 \qquad ( 2 - 3 )$$

$$\frac{{T}_{p}}{{T}_{m}} = {N}^{2} \qquad ( 2 - 4 )$$

Lₚ, Lₘ-width of prototype foundation, width of model foundation, m;
Hₚ, Hₘ-Prototype foundation depth, model foundation depth, m;
σₚ, σₘ-Prototype foundation soil pressure, model foundation soil pressure, kPa;
Tₚ, Tₘ-Prototype consolidation time, model consolidation time, s.

### 2.3 Preparation of Foundation Structure

A uniform layer of oil-based lubricant, such as synthetic grease or industrial butter, is applied to the inner walls of the model box. The sand foundation structure model is prepared in 6 layers, with each layer thickness of 50 mm. In order to ensure the density uniformity of sand, the sand-rain method is used to prepare the model, i.e., according to the pre-calibrated dry density-drop distance curve of the sand-rain method, the drop distance corresponding to the dry density of 1,548 kg/m3 is determined, and then according to this drop distance, the sand is dropped uniformly from the sand-rain method device to the model box. After each layer is filled, three points are randomly selected for sampling to measure the dry density of the model. The measured values are then compared with the preset dry density of 1,548 kg/m³. If the error exceeds 100 kg/m³, the layer must be refilled. After each layer of the model is filled and smoothed, a groove with an about 5 mm depth is created by removing a corner of the layer on a side adjacent to the observation window; and then uniform fine sand dyed white is filled into the groove to form a color line mark for observation of deformation of the foundation structure. The color line information can be found in Section 2.5.2 below. In the process of filling sand model, it is necessary to place a specific measuring device at the preset position, and the position information of measuring device can be shown in the following contents of 2.5.

The water supply system at the bottom of the model is used to supply water uniformly to the soil of the foundation structure until the preset water level is reached and the water supply is stopped. When the water level in the foundation structure model is lower than the model surface, it is an unsaturated working condition. The water level in the foundation structure model is equal to the surface height of the model, which is a saturated working condition. After the water supply operation is completed, it is necessary to observe whether there is any bubble in the model by the observation window of model box. If any bubble is generated, it is necessary to slightly vibrate or knock the model box to discharge the bubble.

### 2.4 Test Procedure

### 2.4.1 Centrifugal Acceleration Applied to the Test Model

After the test model is prepared, the model box is hoisted and fixed in the centrifuge basket, and each measurer device is installed and tested. After the test is started, the centrifugal acceleration is gradually increased in increments of 5 g per step, in 10 increments, until the centrifugal acceleration increases to the preset target value. The increase of centrifugal acceleration is carried out step by step, and each increase is 5 g, so as to simulate the natural deposition of actual foundation material and the consolidation process under the self-weight condition. This graded loading mode is beneficial to more accurately simulate the real stress level in the prototype foundation structure.

Readings from the earth pressure gauge are taken and analyzed when each acceleration stage reaches its preset value. After the earth pressure gauge reading has stabilized, it continues to run 10 min and then enters the next stage of loading process. After the centrifugal acceleration reaches the preset target value of 50 g, the reading of laser sensor is measured and read to determine the displacement of foundation structure in the vertical direction. Then, a vertical load is applied to the strip-type foundations in stages using a hydraulic device.

### 2.4.2 Vertical Load on Strip Base

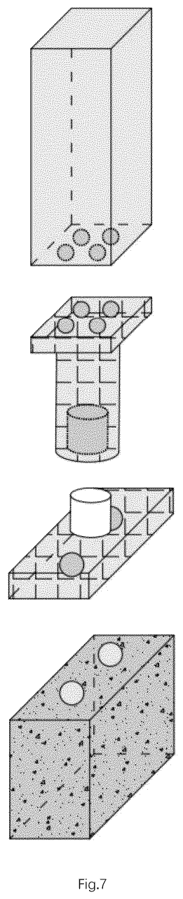

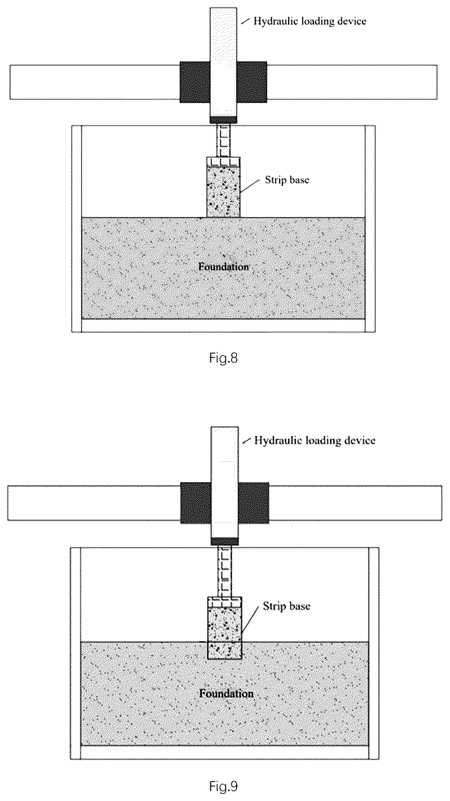

The connection between the hydraulic device and the strip base is as shown in FIG. 6. The hydraulic rod of the hydraulic device is connected to the connecting rod via four bolt holes. The connecting rod is further connected to the hydraulic head via a large bolt. The hydraulic head and the strip base are also rigidly connected using a bolt. The connection method and the structure of the hydraulic device and the hydraulic head are as shown in FIGS. 7 and 8.

The strip base is rigidly connected to the loading device by means of loading screws. The pressing speed of the loading device against the strip base is 0.1 mm/s. The vertical load provided by the hydraulic device is loaded in stages, each stage load is 5 kPa, and the load is maintained for a period of time after the completion of pressurization of each stage load, until the vertical settlement displacement of strip base reaches relative stability, and then the next stage load is applied. The reading of pore water pressure gauge is observed. The relative stability criterion of strip base settlement under each stage load is that the settlement per hour in two consecutive hours is less than 0.1 mm. If the pore water pressure gauge reading exceeds the theoretical hydrostatic pressure, it waits for the pore water pressure gauge reading to fall back to near the theoretical hydrostatic pressure. When the pore water pressure exceeds the theoretical hydrostatic pressure, it indicates that the vertical loading has generated excess pore water pressure within the soil. Maintaining the load while waiting for both the settlement displacement of the strip base to stabilize and the pore water pressure to decrease is essential to ensure the accuracy of the test results. After the vertical displacement of the strip base reaches 50 mm, the loading is stopped for unloading. In the loading process, if it is found that the strip base is inclined and so on, so that the strip base cannot continue to move along the vertical direction, it is necessary to immediately stop pressing the load, and make the test again. In the case of successful test, the state of strip base after the end of loading is as shown in FIG. 9.

The manner in which the strip base and the loading device are rigidly connected ensures that the load can be accurately transferred from the loading device to the strip base and further to the foundation structure. The rigid connection means that the relative displacement between the strip base and the loading device is negligible during the loading process, which ensures the accuracy of the vertical displacement data of the strip base. By trying different loading speed, it is found that 0.1 mm/s is a relatively reasonable loading speed, which can help to more accurately control the loading process and monitor the changes in the readings of various measuring instruments. In addition, the relatively slow loading speed also reduces test errors due to inertial effects, which is advantageous for the test method of the present invention.

### 2.5 Monitoring Scheme

The vertical displacements of the surface of the foundation structure and the strip base are monitored from above the foundation structure by using the laser ranging displacement sensors, and a total of eight sensors are arranged. The internal displacement of the foundation structure is monitored from the side of the model box observation window using a particle image velocimetry (PIV) instrument. Two earth pressure gauges are deployed to monitor the changes in soil pressure within the foundation structure during the increase in centrifugal acceleration. Two pore water pressure gauge are installed to monitor pore water pressure variations during both the centrifugal acceleration increase and the vertical loading of the strip base.

### 2.5.1 Mounting of Laser Ranging Displacement Sensor

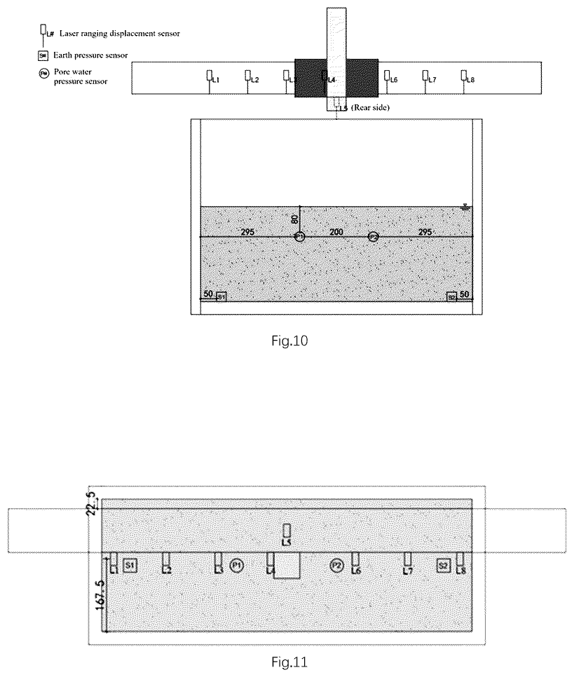

The specific locations of the laser ranging displacement sensors are shown in Table 3 and in FIGS. 10 and 11.

TABLE 3
Position table of laser ranging displacement sensor

| | X coordinate | Y coordinate | Z coordinate |
|---|---|---|---|
| No. | (mm) | (mm) | (mm) |
| L1 | 27.5 | 167.5 | 705 |
| L2 | 147.5 | 167.5 | 705 |
| L3 | 267.5 | 167.5 | 705 |
| L4 | 387.5 | 167.5 | 705 |
| L5 | 425 | 227.5 | 626.5 |
| L6 | 582.5 | 167.5 | 705 |
| L7 | 702.5 | 167.5 | 705 |
| L8 | 822.5 | 167.5 | 705 |

### 2.5.2 Installation of Particle Image Velocimetry (PIV) Equipment

The camera of the particle image velocimetry (PIV) equipment is located at a horizontal distance of 32 cm from the model box, with the height rising 15 cm from a level with the bottom of the model box. According to the light environment and the focal length of the camera, the camera is debugged in order to achieve a clear and complete picture of the camera test model, including the foundation structure, the strip base and a part of the hydraulic rod. The color lines, which are located at the depths of 50 mm, 100 mm, 150 mm, 200 mm and 250 mm from the surface of the foundation structure, are used as markers, and are completed at the stage of filling the foundation structure in layers, respectively. The color line mark color is selected to be white, but other colors may also be selected based on the sensitive hue of the particle image PIV camera, subject to accurate identification. The color line dye should be chosen to be of low water solubility to prevent the dye from flowing and diffusing with the pore water during the water level rise.

### 2.5.3 Installation of Earth Pressure Gauge

The earth pressure gauge positions are shown in Table 4 and in FIGS. 10 and 11.

TABLE 4
Position table of earth pressure gauge

| | X coordinate | Y coordinate | Z coordinate | Maximum |
|---|---|---|---|---|
| No. | (mm) | (mm) | (mm) | range (kPa) |
| S1 | 50 | 152.5 | 0 | 2000 |
| S2 | 800 | 152.5 | 0 | 2000 |

### 2.5.4 Installation of Pore Water Pressure Gauge

The pore water pressure gauge locations are shown in Table 5 and in FIGS. 10 and 11.

TABLE 5
Pore water pressure gauge position list

| | X coordinate | Y coordinate | Z coordinate | Maximum |
|---|---|---|---|---|
| No. | (mm) | (mm) | (mm) | range (kPa) |
| P1 | 295 | 152.5 | 220 | 1500 |
| P2 | 495 | 152.5 | 220 | 1500 |

### 2.6 Record and Use of Monitoring Data

At the beginning of the test, all measuring devices shall be powered on at the same time, so as to ensure that the time zero of reading of each measuring device is the same. The readings of the earth pressure gauge during the increase in centrifugal acceleration are recorded in Table 6. See Table 7 for the reading records of the laser ranging displacement sensor after the foundation structure model reaches the target acceleration and stabilizes before the load is applied to the hydraulic device. See Table 8 for the readings recorded from the individual laser ranging displacement sensors when the hydraulic devices are loaded and when they are loaded.

The load-settlement curve for the strip base is derived from the data recorded in Table 8. In the curve, the load is denoted as q, obtained by dividing the output force of the hydraulic device by the base area of the strip base. The displacement is expressed as s/B, representing the ratio of the actual settlement s to the base width B. According to the Technical Standard for Test of Building Foundation (JGJ 340-2015), the bearing capacity of the foundation structure is determined according to the load-settlement curve as follows.

If the soil around the strip base appears obvious lateral extrusion and the soil around the strip base appears obvious uplift, the upper load value of the phenomenon appears is taken as the ultimate bearing capacity of the foundation structure.

If the settlement of a certain load is 5 times larger than that of the previous one, and the load-settlement curve shows a sharp drop, the previous load value is taken as the ultimate bearing capacity of the foundation structure.

If the settlement of a certain primary load cannot reach a relatively stable standard within 24 hours (namely, the settlement per hour in two consecutive hours is less than 0.1 mm), the previous load value is taken as the ultimate bearing capacity of the foundation structure.

If there is a proportional limit on the load-settlement curve, the load value corresponding to the proportional limit is taken as the ultimate bearing capacity of the foundation structure.

If there is a proportional limit on the load-settlement curve and the ultimate load is reached, half of the ultimate load value is taken as the ultimate bearing capacity of the foundation structure when the ultimate load value is less than twice of the load value corresponding to the proportional limit.

If the settlement displacement of the foundation structure reaches the relatively stable standard after the loading, the proportional limit cannot be determined on the load-settlement curve, and the ultimate load is not reached, the half of the corresponding load value at the completion of loading is taken as the ultimate bearing capacity of the foundation structure.

TABLE 6
Record form of model earth pressure before strip base loading

| | Theoretical | Final |
|---|---|---|
| Current | maximum | stabilization |
| acceleration (g) | pressure (kPa) | pressure (kPa) |
| 5 | 23.31 | |
| 10 | 46.62 | |
| 15 | 69.93 | |
| 20 | 93.23 | |
| 25 | 116.54 | |
| 30 | 139.85 | |
| 35 | 163.16 | |
| 40 | 186.47 | |
| 45 | 209.78 | |
| 50 | 233.09 | |

TABLE 7
Record sheet of model surface displacement
before strip base loading

| No. | Displacement before hydraulic loading (mm) |
|---|---|
| L1 | |
| L2 | |
| L3 | |
| L4 | |
| L5 | |
| L6 | |
| L7 | |
| L8 | |

TABLE 8
Record sheet of pressure and displacement
during loading stage of strip base

| | | | |
|---|---|---|---|
| Time | Hydraulic | Hydraulic | Displacement in |
| (s) | loading | loading | hydraulic |
| | output (kN) | pressure (kPa) | loading (mm) |

Portions of the present invention that are not described in detail can be found in the prior art or are known to those skilled in the art.

The embodiments of the present invention have been described above with reference to the accompanying drawings, but the present invention is not limited to the specific embodiments described above, which are intended to be illustrative only and not limiting. There are many other forms that may be made by those of ordinary skill in the art, inspired by the present invention, without departing from the scope protected by the purposes and claims of the present invention, all of which are within the protection of the present invention.

1. A method for a foundation bearing capacity test based on physical modeling, wherein the method comprises the steps of:
S1, taking a certain amount of foundation soil materials, and a measurer for the test;
S2, preparing a foundation structure to be measured in a model box by the foundation soil materials; fixing each measurer in a predetermined position in or near the foundation structure during the preparation of the foundation structure;
S3, providing a strip base on the surface of the foundation structure or at a certain buried depth;
S4, providing a bearing capacity pressing device in the model box, and rigidly connecting a pressing head of the bearing capacity pressing device to the strip base so as to apply a load to the foundation structure via the strip base during the test;
S5, hoisting and fixing the model box in a basket of a centrifuge, operating the centrifuge according to a pre-set flow to apply a centrifugal force to the model box, and recording the measurement results of each relevant measurer via a data automatic collection system; applying a load to the foundation structure by the bearing capacity pressing device until a target termination condition after reaching a predetermined condition, and automatically recording load-related data; S6, stopping the centrifuge to end the test, and analyzing relevant data recorded during the test to determine the bearing capacity of the foundation structure;
the measurer comprises a plurality of laser ranging displacement sensors, a set of particle image velocimetry equipment, a plurality of earth pressure gauges and a plurality of pore water pressure gauges;
the laser ranging displacement sensor is provided above the foundation structure and is configured for measuring displacement data on the surface of the foundation structure;
the particle image velocimetry equipment is positioned directly opposite the foundation structure, and image data of the foundation structure is collected by an observation window of the model box and a color line mark is identified, so as to realize the measurement of soil body displacement velocities at different positions of the foundation structure;
the earth pressure gauge is provided at the bottom of the foundation structure for measuring the earth pressure of the foundation structure under corresponding test conditions;
the pore water pressure gauge is provided inside the foundation structure, and is configured for monitoring the change of pore water pressure in the process of increasing centrifugal acceleration and in the process of pressing and loading by the bearing capacity pressing device;
in the step S5, according to the contents of operating the centrifuge to apply a centrifugal force to the model box according to a predetermined flow, at the beginning of the test, the centrifugal acceleration is increased step by step according to the increment of n times of gravity acceleration, and the centrifugal acceleration is loaded several times to the preset target value, n being a positive integer; after the acceleration of each stage is loaded to a set value, the earth pressure gauge in the measurer is read and analyzed; after the reading of the earth pressure gauge is stable, it continues to operate for a predetermined period of time, reading the reading of the laser ranging displacement sensor in the gauge, and determining the surface displacement of the foundation structure in the vertical direction; and the above steps are cycled until the pre-set centrifugal acceleration target value is loaded.

2. The method for the foundation bearing capacity test based on physical modeling according to claim 1, wherein when the foundation structure is prepared in the step S2, the foundation structure is prepared by filling the foundation structure in a plurality of layers in order from bottom to top; after each layer is filled and smoothed, a groove with a 5 mm depth is created by removing a corner of the layer on a side adjacent to the observation window; and then uniform fine sand dyed white is filled into the groove to form a color line mark for observation of deformation of the foundation structure.

3. The method for the foundation bearing capacity test based on physical modeling according to claim 2, wherein after the foundation structure is prepared, water is uniformly supplied from the bottom of the foundation structure by a water supply system until the water level in the foundation structure reaches a predetermined water level to simulate different ground water level conditions.

4. The method for the foundation bearing capacity test based on physical modeling according to claim 1, wherein, in the step S5, according to the content of applying a load to the foundation structure by the bearing capacity pressing device,
the bearing capacity pressing device performs a pressing operation by intermittently pressing for a plurality of times, each pressing applied on the strip base provided on the foundation structure at a first pressing speed; after pressing to a first pressure threshold value, the pressing head stops pressing until the measured value of the pore water pressure gauge recovers to the value before pressing, and then continues to press in the above-mentioned manner until the pressing operation ends when the vertical displacement of the strip base reaches a first displacement threshold value.

5. A device for a foundation bearing capacity test based on physical modeling, wherein the foundation bearing capacity test method based on physical modeling as claimed in claim 1 can be realized; the test device comprises:
a model box provided in a basket of the centrifuge and configured for providing a model space of the foundation structure and a measurement space of a plurality of measurers for the foundation bearing capacity test;
a foundation structure provided inside the model box and configured for a model to be tested for foundation bearing capacity test;
a strip base provided on an upper surface or at a certain buried depth of the foundation structure, being in contact with and connected to the foundation structure, and configured for bearing the load applied by the pressing device and transmitting same to the foundation structure, so as to realize the foundation bearing capacity test;
a bearing capacity pressing device which is rigidly connected to the strip base and configured for providing a load for the foundation bearing capacity test;
a centrifuge configured for fixing the model box and applying a centrifugal force to the foundation structure therein, so as to simulate a measurement environment of a prototype foundation equivalent to several times of the self-weight of the foundation structure; and
a plurality of measurers respectively provided inside or around the foundation structure for measuring various parameters of the foundation structure before the test, during the test and after the test is finished;
wherein the foundation structure is prepared in layers, and a dyed sand marker band is provided between each layer;
according to different water level control conditions in the foundation structure, the foundation structure is a sand foundation or a viscous foundation under a saturated working condition, or the foundation structure is a sand foundation or a viscous foundation under an unsaturated working condition; and
the relationship between the pre-set target value of the centrifugal acceleration of the centrifuge and each parameter of the foundation structure comprises:
$$\frac{{L}_{p}}{{L}_{m}} = N$$
$$\frac{{H}_{p}}{{H}_{m}} = N$$
$$\frac{{σ}_{p}}{{σ}_{m}} = 1 ;$$
wherein, in the formula,
N represents that the pre-set target value of centrifugal acceleration is N times of the gravity acceleration;
Lₚ and Lₘ are a base width of the prototype foundation and a base width of the foundation structure, respectively;
Hₚ and Hₘ are a foundation depth of the prototype foundation and a foundation depth of the foundation structure, respectively; and
σₚ and σₘ are a foundation soil pressure of the prototype foundation and a foundation soil pressure of the foundation structure, respectively.

6. The foundation bearing capacity testing apparatus based on physical modeling according to claim 5, wherein the measurer comprises a plurality of laser ranging displacement sensors, a set of particle image velocimetry equipment, a plurality of earth pressure gauges and a plurality of pore water pressure gauges;
the laser ranging displacement sensor is provided above the foundation structure and is configured for measuring displacement data on the surface of the foundation structure;
the particle image velocimetry equipment is positioned directly opposite the foundation structure, and image data of the foundation structure is collected by an observation window of the model box and a color line mark is identified, so as to realize the measurement of soil body displacement velocities at different positions of the foundation structure;
the earth pressure gauge is provided at the very bottom of the foundation structure for measuring the pressure which the foundation structure has under the corresponding environment; and
the pore water pressure gauge is provided inside the foundation structure, and is configured for monitoring the change of pore water pressure in the process of increasing centrifugal acceleration and in the process of pressing and loading by the bearing capacity pressing device.
